(19) United States
(12) Patent Application Publication
(10) Pub. No.: US 2010/0131000 A1
(43) Pub. Date: May 27, 2010

(54) SYSTEM AND METHOD FOR REMOVAL OF MATERIAL FROM A BLOOD VESSEL

(75) Inventors: Richard M. DeMello, Stow, MA (US); Jonathan R. DeMello, Stow, MA (US); Richard R. Heuser, Phoenix, AZ (US); Maureen A. Finlayson, Stow, MA (US)

Correspondence Address: CESARI AND MCKENNA, LLP, 88 BLACK FALCON AVENUE, BOSTON, MA, 02210, US

(73) Assignee: Radius Medical Technologies, Inc., Acton, MA (US)

(21) Appl. No.: 12/275,822

(22) Filed: Nov. 21, 2008

(51) Int. Cl. A61B 17/221

(52) U.S. Cl. 606/200; 29/428

(57) ABSTRACT

This invention provides a device and controllably expansive tool tip for thrombus removal. According to one embodiment, the controllably expansive thrombus removal tool tip (e.g., a screen and/or mesh) may be collapsed by pushing on an actuating handle, and then advanced into a balloon or guiding catheter until a distal end of device has reached the thrombus. The tool tip is then expanded by pulling the actuating handle backward; and the radially extended (expanded) tool tip is moved to receive and substantially surround (e.g., encompass) the thrombus. The tool tip may then be collapsed again by pushing on the actuating handle to engage (tighten around) the thrombus, and the device may be withdrawn from a patient's body through the vascular system with the thrombus engaged by the tool tip.

### CROSS-REFERENCE TO RELATED APPLICATIONS

The present application is related to commonly assigned copending U.S. patent application Ser. No. 12/098,201, which was filed on Apr. 4, 2008, by Richard M. DeMello, et al. for a SYSTEM AND METHOD FOR REMOVAL OF MATERIAL FROM A BLOOD VESSEL USING A SMALL DIAMETER CATHETER, which is a continuation-in-part of commonly assigned copending U.S. patent application Ser. No. 11/583,873, which was filed on Oct. 19, 2006, now published as U.S. Publication No. US2007-0118165 on May 24, 2007, by Jonathan R. DeMello, et al. for a SYSTEM AND METHOD FOR REMOVAL OF MATERIAL FROM A BLOOD VESSEL USING A SMALL DIAMETER CATHETER, which is a continuation-in-part of U.S. patent application Ser. No. 11/074,827, which was filed on Mar. 7, 2005, now published as U.S. Publication No. US2005-0234474 on Oct. 20, 2005, by Richard M. DeMello, et al. for a SMALL DIAMETER SNARE, which claims the benefit of U.S. Provisional Patent Application Ser. No. 60/551,313, which was filed on Mar. 8, 2004, by Richard M. DeMello et al., for a SMALL-DIAMETER SNARE, each of which are hereby incorporated by reference.

### BACKGROUND OF THE INVENTION

1. Field of the Invention

The present invention relates to surgical catheters, and more particularly to devices for removing thrombus, and other blockages and materials from blood vessels.

2. Background Information

Certain snare and similar devices have become available over recent years for retrieving malfunctioning or misplaced devices or blockages such as plaque and thrombus within the cardiovascular and non-vascular regions of the body. These typically consist of fairly large diameter sheaths, which house a movable central wire or wires whose distal ends are formed into a loop, plurality of loops or other purpose-built shape. The loop is used to ensnare and capture the desired object for withdrawal and removal from the body, while other shapes may be used to grasp or capture softer biological materials. In use, the snare or another distal tool is typically passed through a guiding catheter or other introducing catheter that is placed within the vasculature and is directed to the vessel or area where the misplaced or malfunctioning device is located. The snare/distal tool can then capture the intended device or material and retrieve it out of the body through the introducing catheter or by withdrawing both the snare and the introducing catheter in tandem.

Currently available snares and similar distal tools are generally designed using large diameter outer sheaths that require relatively large entry sites. This may result in complications such as excessive bleeding and/or hematomas. Additionally, because of the large diameter, it may be necessary to remove the existing catheters and exchange to other larger devices, resulting in an increase in the overall time and cost of the procedure. A third disadvantage of the old means is that the outer sheath, which is typically made of a plastic material, exhibits little or no torque control, which can make ensnaring the misplaced or malfunctioned device or removing other materials very difficult. Lastly, because of the size and stiff design of these snare/distal tool devices, they have a very sharp distal leading edge which cannot be safely advanced into small diameter vessels such as those in the coronary and cerebral vasculature without risking damage to the vessel wall.

An exemplary small-diameter snare design that overcomes many of the concerns above is provided in commonly owned U.S. Pat. No. 6,554,842, entitled SMALL DIAMETER SNARE by Heuser, et al., the teachings of which are expressly incorporated herein by reference. Devices, such as the exemplary Heuser design, are characterized by a small-diameter outer sheath that has a relatively thin wall (for example, approximately 0.0020 inch or less in wall thickness) so as to accommodate an axially movable/rotatable central core wire of approximately 0.008 inch. The structure allows a snare loop attached to the distal end of the core wire and housed within the open distal end of the sheath to be selectively extended from the sheath end, withdrawn and torqued. This sheath is at least partially composed of metal. However, the thinness of the tube, and its metallic content make it susceptible to splitting, fracturing and fatigue failure under stress. In addition, the metal section of the tubular outer sheath tends to experience permanent (plastic) deformation when bent, and once deformed, the central core wire will tend to bind upon the lumen of the sheath, rendering the device inoperable for its intended purpose. In addition, the outer wall of the metal tube section has a lubricious coating, such as PTFE (Teflon), which is typically approximately 0.0010 inch in thickness. This necessitates further downsizing of the sheath overall outer diameter thereby reducing the inner diameter available for accommodating the central core wire and at the same time increasing the risk of inadvertent failure of the device through breakage or plastic deformation.

Further considerations arise in the case of a non-snare device used to remove materials from blood vessels. Within the U.S. alone, approximately 700,000 strokes occur every year. The majority of these (83%) are ischemic strokes due to blood clots (thrombus) that become lodged in and block cerebral vessels. It has been documented that if the blockage can be eliminated within a short period of time (up to 8 hours), the patient can experience a full recovery from the stroke. Presently, clot-dissolving drugs can be administered to break up the clot and restore blood flow, however these drugs must be administered within 3 hours of symptom onset as they take considerable time to become effective. Unfortunately, not all patients are medically eligible to receive these drugs and even those who otherwise are eligible often do not arrive for medical treatment within the 3 hour limit. In these patients, mechanical removal of the blood clot has been shown to have a significant positive outcome for such patients.

Several devices have been designed to break up and suction-out thrombus in the large vessels of the legs and coronary arteries. These use a variety of therapeutic means to accomplish the breaking up, such as water jets, mechanical maceration, ultrasound or photo-acoustic shock waves, and laser ablation. All of these devices, however, have limitations when working in the cerebral vessels. First, they tend to be large and bulky and very difficult or impossible to navigate above the skull base. Secondly, their therapeutic means can be extremely vigorous, resulting in damage to the delicate blood vessels in the brain. In use, the devices require removal of an already placed microcatheter, and the insertion of a replacement catheter that accommodates the device.

One such device that is used to treat blood clots is a mechanical capture device whereby the blood clot is grasped and pulled out of the distal vessels of the brain. The MERCI retrieval device (available from Concentric Medical of Mountain View, Calif.) is a 0.014-inch guidewire that can be passed through a catheter and into the blood clot as a straight wire and then can be remotely shaped into a corkscrew configuration (e.g., by extending the straight wire out of a surrounding sheath, thus “springing” into the pre-shaped corkscrew), becoming intertwined within the blood clot. The wire is then withdrawn from the distal cerebral vessel pulling the blood clot with it. Although this device addresses the ability to navigate above the skull base, it has one major shortcoming, namely, the corkscrew segment of the wire must be very soft and flexible in order to navigate within the brain. This reduces the ability of the device to remain in the cork-screw shape as it is withdrawing the blood clot. During withdrawal, the wire can straighten and the blood clot can be partially or fully released resulting in greater injury to the patient through thromboembolism. A more effective tool for removal of thrombus reduced risk of release or breakup and the ability to navigate smaller blood vessels is highly desirable.

### SUMMARY OF THE INVENTION

This invention overcomes prior disadvantages by providing a device (e.g., a small-diameter device) for removing thrombus and other materials from vascular lumens consisting of a hollow, elongate (e.g., thin-walled) outer sheath, a core/actuating wire, and a capture mechanism. The sheath may be constructed from polymer, e.g., at least at a distal part thereof for enhanced flexibility and can be metal at an adjoining proximal part for added strength. A single central core wire extends through the entire length of the sheath. The outer diameter of the core wire is sized close to the inner diameter of the sheath while allowing for axial sliding, in order to maximize the support to the body portion of the snare device. The distal end of the core wire has a tapered section of reduced diameter or cross section to provide a “guidewire-like” flexibility to the distal portion of the device. Also, a tool tip (or “capture segment”) for removal of thrombus is provided at the distal end of the sheath and core wire that can be controllably expanded to engage a thrombus and remove the thrombus from the blood vessel. In particular, the controllably expansive capture segment may illustratively comprise a braided or meshed screen-like material adapted to open and close around a thrombus.

In use, the controllably expansive thrombus removal tool tip is collapsed by pushing on the actuating handle. The device is then advanced into a balloon or guiding catheter until the distal end of the core wire has reached (exited) the distal end of the thrombus. The tool tip is then expanded by pulling the actuating handle backward; and the radially extended (expanded) tool tip is moved to receive and substantially surround (e.g., encompass) the thrombus. The tool tip may then be collapsed again by pushing on the actuating handle to engage (tighten around) the thrombus, and the device may be withdrawn from the patient's body through the vascular system with the thrombus engaged by the tool tip.

### BRIEF DESCRIPTION OF THE DRAWINGS

The invention description below refers to the accompanying drawings, of which:

FIG. 1 is a partial side cross section of a device according to an illustrative embodiment of this invention;

FIG. 2 is a partial side cross section of the device including a manipulator handle assembly attached to the proximal end thereof;

FIG. 3 is a full cross section in the region of the slide actuator of the handle, taken along line 3-3 of FIG. 2;

FIGS. 4A-B illustrate a controllably expansive tool in an expanded orientation for removal of thrombus and other materials according to an illustrative embodiment of this invention;

FIG. 5 illustrates an embodiment of the controllably expansive tool in a collapsed orientation according to embodiments of this invention;

FIGS. 6A-C illustrate another embodiment of the controllably expansive tool according to embodiments of this invention;

FIG. 7 illustrates another embodiment of the controllably expansive tool according to embodiments of this invention; and

FIGS. 8A-D illustrate a blood vessel and removal of a thrombus therein by a controllably expansive tool according to embodiments of this invention.

### DETAILED DESCRIPTION OF AN ILLUSTRATIVE EMBODIMENT

A. Thrombus Retrieval Device and General Design Details

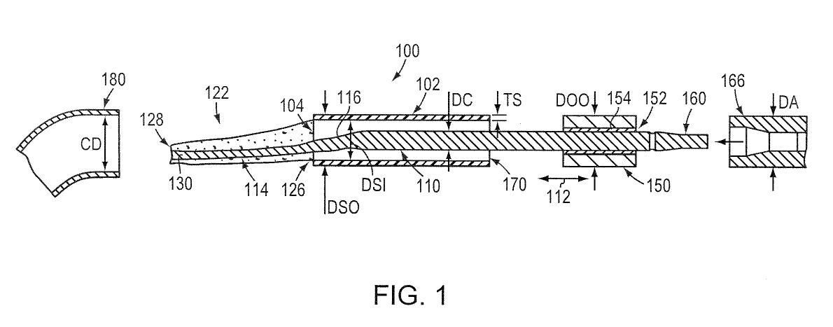

FIG. 1 shows an example (e.g., small diameter) thrombus retrieval device (or snare device) 100 according to an embodiment of this invention. Illustratively, the device 100 includes of a hollow, elongate, thin-walled polymer outer sheath 102. The sheath 102 may include a radiopaque marker located at or adjacent to the open distal end 104 for visualization under fluoroscopy. The polymer can be any one of a number of acceptable biocompatible polymers with sufficient structural strength to support a thin-walled (approximately 0.0020 inch maximum wall thickness TS) structure without rupture or other failure under normal use conditions. Alternatively or in addition, the thin-walled outer sheath 102 may be made from a metal tube, a metal spring coil with an outer polymer jacket, or a combination of a metal tube proximal portion and a thin-walled polymer tube distal portion (described below).

In one embodiment, the sheath is constructed from polyimide with a tungsten filler for radiopacity. The radiopaque filler may be added to the sheath polymer during processing, or a radiopaque material may be added to the outer surface via vapor deposition, plating, ion implantation processes, or the like. Alternatively, radiopaque markers can be applied at the distal end and/or other known locations along the sheath, and thus, an overall tungsten filler/radiopaque coating can be omitted. As discussed further below, the outer surface can include thereon a polytetrafluoroethylene (PTFE or “Teflon”) coating upon some, or all, of its outer surface for enhanced lubricity. Alternatively, the outer sheath coating can be constructed form a hydrophilic material that provides lubricity, instead of a PTFE coating. The sheath polyimide material is commercially available for a variety of vendors and sources and is becoming accepted in a variety of medical device applications. It has the property of allowing a very strong, thin-walled cylindrical-cross section tube to be made therefrom, with wall thicknesses on the order of approximately 0.00075 inch to 0.010 inch in normal applications. Nevertheless, the resulting polyimide tube can withstand high pressures in excess of 750 PSI when employed in the size range of the sheath of this invention. Polyimide also resists high temperatures, as much as 1000 degrees F., or greater. Accordingly, polyimide is desirable as a sheath material based upon all of the above-described superior performance characteristics. Nevertheless, it is expressly contemplated that other equivalent plastic/polymer materials suitable for forming a thin-walled sheath tube with similar or better properties (e.g. high strength, thin wall-thickness limits, small diametric sizing) may also be employed as an acceptable “polymer” herein.

The outer sheath 102, which forms the main support and outer framework of the device 100 has an overall length sufficient to traverse the body's varied vasculature, and is (for most applications) permissibly in a range of between approximately 20 cm and 500 cm (more typically between 120 cm and 300 cm), such as depending upon (among other factors) the location of the insertion point into the body cavity/vasculature, and the location of the target thrombus, or other material, to be acted upon by the device. The outer diameter DSO of the sheath is, for most applications, in a range of between approximately 0.010 inch to 0.045 inch (more typically between 0.010 inch and 0.021 inch), although the diameter may fall within the range of 0.008 inch to 0.250 inch. In general, where the outer diameter is less than 0.35 inch, the device 100 may fit easily through a standard balloon catheter.

A single central core wire (or “actuating wire”) 110 extends through the entire length of the sheath 102. The outer diameter DC of the body of the core wire is sized close to the inner diameter DSI of the sheath while allowing for axial sliding (double arrow 112), in order to maximize the support imparted by the core wire 110 to the body portion/sheath 102 of the snare device 100. (For instance, example guidewire dimensions are 0.014 inch and 0.035 inch diameters.) The distal end 114 of the core wire 110, however, may have a tapered section 116 of reduced diameter or cross section to provide a “guidewire-like” flexibility to the distal portion of the device. According to one or more illustrative embodiments, a tool tip or capture segment 122 (shown collapsed) may extend from the end of the sheath configured in a manner described herein so as to increase the ability to ensnare and capture objects (e.g., a thrombus). Capture segment 122 may be attached at a proximal end 126 to the outer sheath 102, and at a distal end 128 to the distal-most portion 130 of the central core wire 110, as described further below. For example, depending upon its materials and configuration, the capture segment 122 may be attached at its ends via welding, soldering, brazing (or other high-strength metal-flowing means), adhesives, wrappings, sewing, etc. The core wire 110 may be further secured slideably to the outer sheath (e.g., through a loop, not shown) in order to maintain the core wire's orientation to one side of the capture segment 122.

The central core wire 110 may be made from metal for flexibility and strength. In one embodiment, the central core wire 110 may be made by connecting a proximal stainless steel portion, for support and stiffness, to a distal nitinol portion, for torqueability and kink resistance. Likewise, it can be made from 300-series stainless steel or a stronger, heat settable material such as 400-series stainless steel, alloy MP35N, a chromium-cobalt alloy such as Elgiloy, or nitinol in its super elastic or linear elastic state.

Note, because a thin-walled polymer sheath is illustratively employed, it advantageously allows for a maximized central core wire diameter, which in turn, provides stiffness for torque control and axial pushability of the device. In this and other embodiments described herein, however, the sheath can be all polymer along its entire length, or can be constructed from a combination of polymer and metal. For example, the distal part of the sheath can be the above-described polyimide material (or another appropriate polymer), while the proximal part can be constructed from 300-series stainless steel or any other appropriate metal. This affords the desired flexibility in the distal part, while providing greater strength and rigidity against buckling in the proximal part. Flexure is required less and beam strength (so as to assist in driving the device distally) is required more in the proximal part. The distal part may be joined to the proximal part at a joint (not shown) located at a predetermined distance along the device. The joint can be accomplished using adhesive or any other acceptable joining technique. In one example, the polymer distal part is approximately 40 centimeters in length, while the metal proximal part is approximately 140 centimeters in length. These measurements are widely variable depending upon the overall length of the sheath, the purpose of the device (e.g. where it will be inserted) and the distance of the distal part in which high flexibility is required.

After assembly of the core wire 110, its insertion into the sheath 102, and the attachment of the capture segment 122, a second short, hollow tube may be fitted over the proximal end 152 of the central core wire 110 and attached thereto by a filler or adhesive 154 to provide an actuating handle 150 so as to slideably move the central core wire axially (as indicated by double arrow 112) within the sheath 102, thus selectively expanding and contracting the capture segment 122 at the open distal end 104 of the sheath 102. In one embodiment, the actuating handle 150 may be sized with an outer diameter DOO similarly (or identically) in outer diameter DSO to the main body of the sheath 102. The exposed proximal end 152 of the core wire 110 may include a narrowed-diameter end 160, with a special connection so that an additional length of wire 166 can be attached to it, thereby extending the overall length of the snare device. This extension may have a similarly sized outer diameter DA to that of the handle 150 (DOO) and sheath 102 (DSO). The attachment of this similarly small-diameter extension allows for the exchange of one catheter for another catheter over the body of the snare (and extension). The entire device when complete (including the actuating handle 150) can be made less than 0.014 inch in overall outer diameter, and is therefore capable of being placed directly through a PTCA balloon catheter or other small-diameter catheter 180, having a sufficiently large inner diameter CD, that may already be in place within the patient (e.g. CD>DSO). Since the actuating handle is equally small in diameter, it also passes through the small-diameter catheter with an extension piece joined behind the handle to the attachment end 160, and thereby allowing the device to be guided even deeper into the patient when needed. The capture segment and device may also be passed through the guiding catheter along side of the balloon or access catheter without the need to remove the prior device and, thus, lose temporary access to the site within the patient. For example, the device 100 may be initially passed through the PTCA balloon catheter, which is already located within the target area. The balloon catheter can then be removed and replaced with a larger-inner diameter catheter to allow removal of the object (e.g., thrombus).

The actuating handle 150 may consist of a metal or a polymer tube. In an alternate embodiment (not shown) the actuating handle may consist of a tube slideable within a second metal tube that is attached to the proximal end 170 of the sheath to maintain an axial orientation between the proximal end of the core wire 102 and sheath, thereby minimizing permanent bending or kinking of the core wire at or near this proximal location.

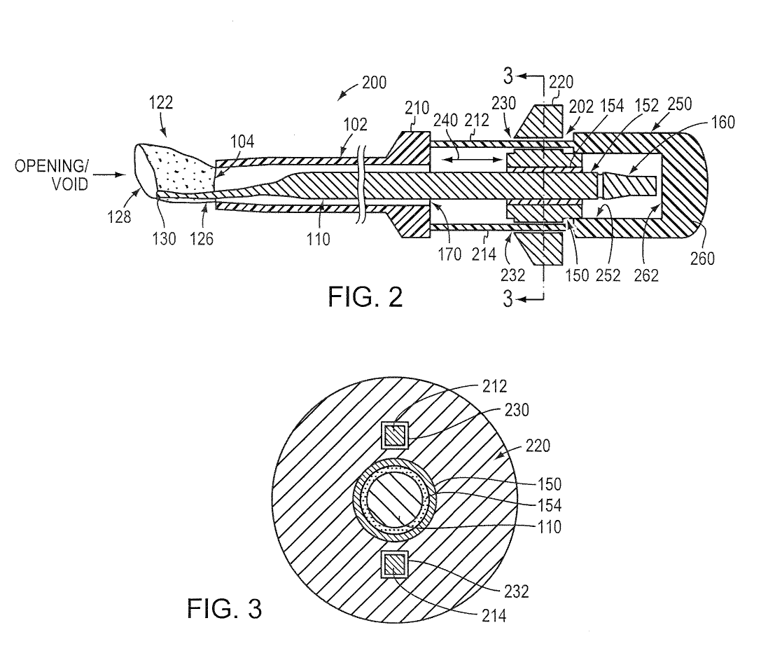

While the depicted actuating handle 150 is of similar outer diameter as the sheath 102, it is expressly contemplated (where the handle will not be passed into another catheter) that the actuating handle may be made in a diameter significantly larger than the snare device so that it may also serve as a torquing handle, similar to those utilized in routine small-diameter guidewire placement. FIG. 2 shows an overall version 200 of the device that includes an enlarged handle attachment 202 attached to the previously described snare device 100 of FIG. 1 (with like components in FIGS. 1 and 2 retaining like reference numbers, capture segment 122 now shown expanded). The handle attachment 202 extends over the actuating handle 150, as discussed in more detail herein. The handle attachment 202 may be made from a polymer material which (in an embodiment of this invention) is injection molded and mechanically attached onto the device or (in another embodiment) may be over molded directly onto the device.

The handle attachment 202 includes a base ring 210 that is secured to the outer surface of the proximal end 170 of the sheath 102. In a detachable-handle embodiment, the ring can consist of a conventional lockable collet structure in which turning of an outer element reduces the diameter of an inner locking element to deliver securing hoop stress to the distal end 170 outer surface of the sheath 102. The base ring is connected to two or more ribs 212 and 214 that are also shown in cross section in FIG. 3. The ribs have a square or rectangular cross-section.

A second actuating ring 220 is secured onto the actuating handle 150 either permanently or detachably. Where it is detachable, the ring may also utilize a locking collet structure (not shown) as described above. The ring 220 includes at least two apertures 230 and 232 to allow passage of the respective ribs 212 and 214 through the ring, such that the ring 220, actuating handle 150 and core wire 110 can slide axially (double arrow 240) to move the core wire with respect to the sheath 102. The ribs, with their square or rectangular cross-section prevent rotation of the ring 220 and interconnected core wire 110 and handle 150 relative to the sheath. The connection is sufficiently strong so that rotation of the handle assembly 202 causes torquing of the entire device so as to rotate the capture segment 122 (in one or more embodiments) into a desired rotational orientation. In an alternate embodiment, the ring 220 may have a non-circular cross-section. In another alternate embodiment (also not shown), the ring 220 may also allow at least limited rotation of the core wire relative to the sheath by utilizing arcuate slots at the ribs.

The handle assembly 202 includes a rear gripping member 250 that connects to the proximate ends of the ribs 212 and 214. The gripping member remains outside the body and is sized to provide an ergonomic hand piece for a practitioner during a procedure. In one embodiment the member 250 has an outer diameter of approximately ½ to ¾ inch and an external length of approximately 4 to 5 inches. However, it is expressly contemplated that both these dimensions are widely variable outside the stated ranges herein. The member 250 defines an inner cylindrical barrel 252 having an inner diameter sized to slideably receive and guide the proximal end of the actuator handle 150. The barrel 252 is sufficiently long so that its inner end wall 262 (of end cap 260) is not struck by the end 160 of the device at maximum withdrawal (as approximately shown) of the core wire 110 into the sheath 102 (for expansion of capture segment 122, as described below).

Coatings can be applied to the outer surfaces of each of the core assembly and the sheath assembly to reduce friction between the core and the tube as well as to enhance movement of the retrieval device within a catheter. In one embodiment, a lubricious coating, such as PTFE (Teflon), hydrophilic, or diamond-like coating (DLC) may be applied to the outer surface of the sheath to reduce friction. Likewise, one of these coatings may be applied to the outer surface of the core wire to reduce friction with respect to the sheath. Since the coating adds a quantifiable thickness to the thickness of the sheath and/or diameter of the core wire, the overall size of components should be adjusted to compensate for the thickness of any lubricating coating. For example, the outer diameter of the sheath may need to be reduced to maintain a desired 0.035-inch or less outer diameter. Likewise, the thickness of the uncoated wall of the sheath may be reduced to maintain the desired inner diameter and create a final wall thickness, with coating, of approximately 0.0020 inch.

B. Controllably Expansive Thrombus Retrieval Device

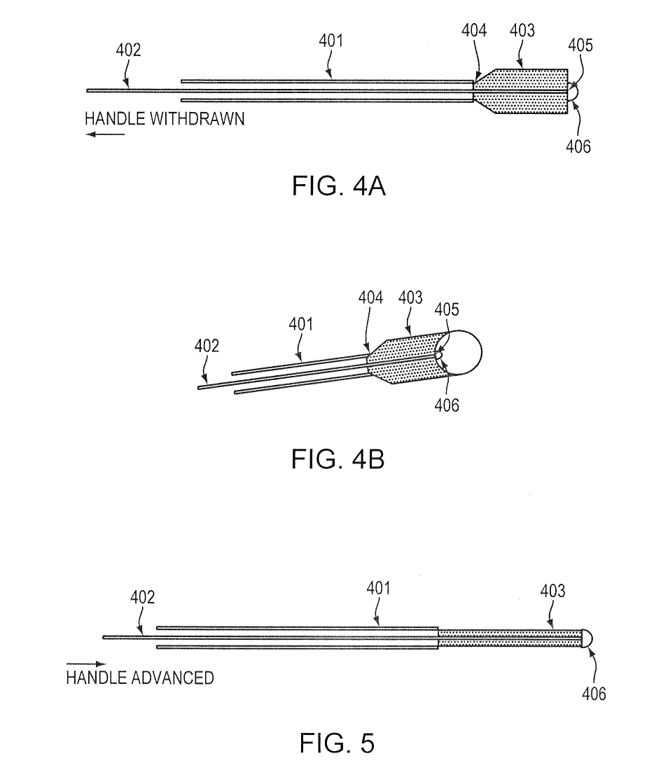

Having described the general structure of the device, a more specific description of the braided mesh/screen capture segment is now described with reference to FIGS. 4A-5 (hereinafter referenced as device 400). The device 400 comprises an outer sheath 401, a core/actuating wire 402, and a capture segment 403. The capture segment 403 is shown in an expanded or “open” state in FIGS. 4A-B, and in a collapsed or “closed” state in FIG. 5. An additional distal cap 406, located at the distal end of core wire 402, may be rounded to form an atraumatic leading edge to facilitate movement through the blood vessels without causing damage. Illustratively, the capture segment 403 may be formed from a braided, metallic material. For example, suitable materials for screen 403 may comprise nitinol, stainless steel, or cobalt-chromium alloy, although it is conceivable that the braid/screen could also be made from a non-metallic material such as a cloth or polymer fibers. Note that a portion of or the entire mesh/screen segment 403 may be made radiopaque to aid in fluoroscopic visualization, such as by adding marker bands (e.g., at a distal end), electroplating, vapor deposition, or ion-beam bombardment/implantation of a radiopaque material onto the mesh. Also, the screen 403 may be made from a radiopaque material, such as platinum-tungsten wire (e.g., typical of guidewire coils).

The proximal end of the braided capture segment 403 may be attached about its periphery to the distal end of the outer sheath 401, at point 404, and the distal end of the braided capture segment may be attached at (or along) one side to the distal end 405 of the actuating wire 402. The distal end of the capture segment thus remains open when the capture segment is in its expanded state, thus creating a void within the capture segment for receiving a thrombus or other material. For example, the mesh/screen material may, though need not, be pre-formed, such by heat setting or other process (e.g., pre-bending), to create a desired shape when expanded (and/or when compressed/collapsed) to optimize capturing ability, such as the open shape as shown. Notably, while a single core wire attachment is shown, a plurality of attachments (e.g., for a split core wire 402) may be made, so long as the distal end of the capture segment generally consists of one or more opening that are sufficient in size to capture a thrombus as described herein. Also, while the core wire 402 is shown on the inside of the capture segment 403, other locations may be possible, such as on the outside of the capture segment or within the capture segment (that is, a part of and thus neither inside nor outside the capture segment) as may be appreciated by those skilled in the art.

In operation, as shown in FIG. 5, the core/actuating wire 402 is advanced forward such that the braided/screen material is stretched longitudinally causing the body of the braided section to collapse downward against the actuating wire. (For example, this action is similar to that of a known “Chinese finger trap.”) In other words, FIG. 5 shows one embodiment of the controllably expansive capture segment 403 for a thrombus retrieval device that is in a collapsed orientation/position. When the core/actuating wire 402 is withdrawn backward (FIGS. 4A-B), the braided capture segment 403 returns to its expanded shape, whereby a distal most end of the capturing mechanism is in an open (net-like) configuration.

In particular, for controlling expandability operation of this embodiment, when the actuating/core wire 402 is advanced forward, as shown in FIG. 5, the braided mesh/screen capture segment 403 collapses downward against the core wire (a “collapsed state”) so that the device can be introduced into the body/vessel (e.g., with an outer diameter substantially close to that of the outer sheath 401) and directed to the thrombus location. When the core wire 402 is withdrawn within the outer sheath 401 (as in FIGS. 4A-B), the capture segment 403, attached or otherwise restricted from entering the outer sheath 401, expands outwardly (an “expanded state”) to the desired shape. The capture segment may then be moved into position to surround (encompass) a thrombus. The capture segment thus envelops the thrombus/material, which remains generally intact. Also, as described below, the capture segment 403 may again be collapsed around the surrounded thrombus, by advancing the core/actuating wire 402. Various techniques may be used to lock or otherwise secure the core wire in position to maintain the capture segment in either the expanded or collapsed state to allow an operator of the device to securely position the capture segment in one or the other state without having to manually apply consistent force (tension) to the actuating/core wire.

As noted, in a collapsed state, the OD of the capture segments may be sized substantially similar to the OD of outer sheath itself (e.g., no greater than an ID of a catheter in which the outer sheath/device is meant to traverse). In an expanded state, the capture segment extends to approximately the vessel diameter so that it may be used to capture a thrombus or other material within the vessel. The capture segment may then be collapsed to move the thrombus/material, e.g., removing it from the vessel or otherwise repositioning the thrombus/material to another location. The range of radial extension of a fully-deployed tool tip or other capture segment is highly variable. It can be anywhere from 1 millimeter to 100 millimeters in various embodiments. This radial sizing depends partly upon the size of the space into which the capture segment is being inserted. More typically, a capture segment will have maximum radial extension between approximately 2 millimeters and 35 millimeters. In other words, the radial projection (RT) of the distal tool tip should be sufficient to surround the approximate dimension of the thrombus/material to be cleared, yet remain smaller than the inner diameter of lumen of any vessel through which the deployed tool is expected to carry. This helps to reduce the chance of injury to vascular walls. The distal-to-proximal length (LT) is also highly variable, depending upon the position of the core wire, and the current and/or desired radial projection.

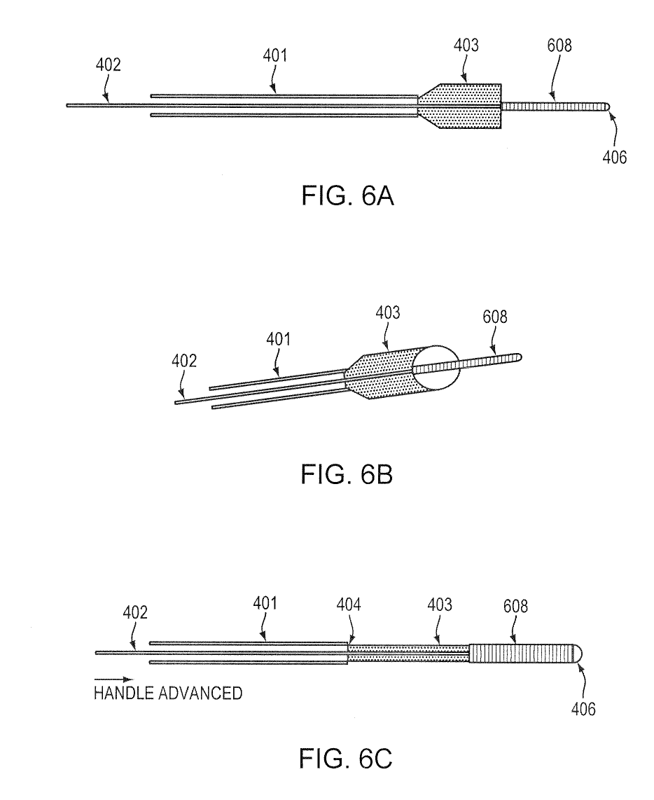

Notably, in alternative or additional embodiments a distal atraumatic spring portion may be added to the distal end of the core wire to facilitate movement through the blood vessels without causing damage. In particular, as shown in FIGS. 6A-C, the core wire 402 may illustratively continue beyond the distal end of the capture segment 403 (e.g., by approximately 1-3 cm), and may taper to a smaller (e.g., more flexible or “softer”) diameter. A radiopaque spring coil 408 fits over the end of the core wire and is secured at its distal end to the distal most portion of the core wire. The proximal end of spring coil 408 is secured to the core wire at the distal end of the capturing segment 403, and thus the proximate end of the extended core wire.

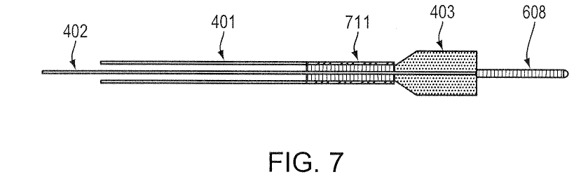

Further, in addition or in the alternative, the outer sheath 401 may contain a flexible coil portion on its distal end. For example, FIG. 7 shows a further embodiment of a thrombus retrieval device in which the outer sheath 401 has a hollow flexible coil segment 711 added onto its distal end. The coil 711 provides a flexibility at the distal end of the outer sheath that aids in negotiation of the device through constricted portions of the anatomy, such as that found in the brain (e.g., often extremely tortuous). Coil 711 is illustratively attached to the distal end of the outer sheath 401 in a manner that the ID of the coil remains open to allow the actuating/core wire 402 to slide therein. In doing so, coil 711 may be considered as an extension of the outer sheath 401. In this embodiment, the capture segment 408 may be attached at its proximal end to the coil 711 and at its distal end to the core wire 402.

In each embodiment described herein, the mesh/screen of the capture segment 403 is straightened out and thus collapsed for insertion of the device into the body when the actuating core wire is advanced, and expanded (e.g., enlarged or opened) when the core wire is retracted/withdrawn. The capture segment 403 may then be re-collapsed (e.g., around a thrombus) by advancing the actuating core wire 402. Also, in use, the actuating wire may be withdrawn or advanced and locked in position as described above, such that the capture segment remains in the capture/collapsed state to remove the thrombus or move the thrombus/material to a desired location, as described below.

Note that each of the tool designs described herein is by way of example. For instance, the size of the capture segment can be varied based upon the size of the target vessel, as well as the size and characteristics of the material being engaged. Further, while the capturing segment is illustratively shown as a symmetric design, the segment 403 may be configured in multiple fixed diameters, or as other pre-configured shapes and/or varying diameters not explicitly shown. These illustrations, therefore, are merely representative, and should not be limiting on the scope of the present invention.

C. Procedures for Withdrawal of Thrombus and Other Materials with Controllably Expansive Capture Segments

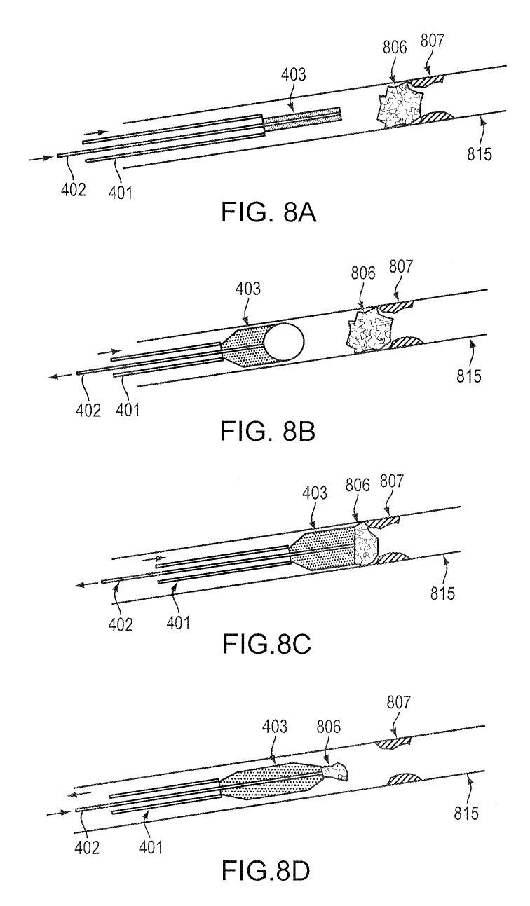

Having described the general structure of the device and more specifically an illustrative braided mesh/screen capture segment, a procedure of removing a thrombus or other material from a blood vessel is now described in further detail. FIG. 8A shows the insertion of the device 400 into a blood vessel 815 containing a thrombus 806 (or other internal blockage, natural or man-made), which is prevented from traversing the vessel 815 by a constriction 807 (e.g., a plaque build up). Initial insertion of the device can be made via the aorta or another suitable blood vessel, where the device 400 may be advanced into a balloon or guiding catheter 410 until the distal end of the device and capture segment 403 has exited the distal end of the catheter. In particular, with the actuating core wire advanced forward, thus collapsing the capture segment 403, the device may be advanced into the vessel 815 and maneuvered (e.g., torqued, manipulated, etc.) into a location adjacent to the object to be retrieved, e.g., a thrombus 806. (Notably, the collapsed capture segment should be constructed and assembled in a manner that reduces drag within the vessel or encapsulating catheter, and prevents snagging/catching along vessel/catheter walls. Also, once deployed, the positions of the core wire 402 with respect to the sheath 401 can be locked using, for example, the above-described handle lock mechanism.)

Once positioned adjacent to the thrombus 806, the core/actuating wire 402 is withdrawn allowing the capture segment 403 to expand into its open state, e.g., substantially sized to the inner diameter of the vessel 815, as illustrated in FIG. 8B. The entire device 400 may then be advanced forward and manipulated such that the capture segment 403 is moved over the thrombus 806, as shown in FIG. 8C. In this manner, the capture segment 403 surrounds (or encompasses or envelops) the thrombus material, which is generally kept intact, since no portion of the capture segment need pierce or otherwise enter the material/thrombus.

Once the thrombus 806 is enclosed within the capture mechanism 403, the core/actuating wire 402 is re-advanced causing the capture mechanism 403 to collapse around the thrombus 806, thereby providing a secure capture of the thrombus/material (FIG. 8D). Generally, the thrombus material may be sufficiently accretive such that it can be grasped and withdrawn without fragmentation, thus avoiding a thromboembolism or another undesirable effect. As such, all or a large portion of the thrombus 806 is captured and can be withdrawn with the device 400 from the patient's body. Alternatively, the thrombus may be retrieved back to a larger area within the body, where the thrombus can be safely aspirated out of the body using a large bore catheter and syringe (not shown).

The capture segment may instead be maintained in its collapsed state, pushed through and into the thrombus so that the capture segment resides at least partially within the thrombus, and then expanded by withdrawal of the core wire, allowing the capture mechanism to expand outward into the thrombus. The device may then be withdrawn, removing the thrombus from the vessel (e.g., from patient's body), while maintaining outward pressure from within inside the thrombus.

The above-described insertion procedures can be modified to accommodate the characteristics of the particular tool tip shape and size. A variety of additional tools and/or internal scanning devices can be employed to facilitate the procedure in accordance with known medical techniques. Note also that the proximal end of the thrombus-removal device described herein includes a proximal end that allows removal of the actuator handle and addition of a small-diameter extension. When the extension is added, the practitioner can pass another catheter over the inserted device sheath, thereby using the device as a guide for the larger diameter catheter.

Notably, the controllably expansive capture segments described above should be substantially sized in its expanded state so that it approximates the vessel diameter. For instance, vessel diameters where such a device may be used can typically range from 1 mm to greater than 35 mm, however, most thrombus retrieval procedures are performed in vessels ranging between 1 mm and 10 mm. In this manner, the capture of the thrombus is assisted by substantially allowing the capture segment to more fully surround and encompass the thrombus.

Further, in accordance with one or more embodiments of the present invention, the capture segment may be advantageously coated with a material to attract a thrombus, such as an ionic charge, or may include brushes and/or filaments (not shown). Also, the capture segment may be coated with a thrombus dissolving drug, such as Integrelin®, ReoPro®, or other thrombolytic agents as will be understood by those skilled in the art. Alternatively or in addition, the device may be constructed with a gap between the outer sheath and the actuating/core wire in order that localized drugs (e.g., thrombolytics) may be infused through the outer sheath and delivered directly to the thrombus.

The thrombus removal devices described herein may also operate to open the impeded vessel to allow blood flow. While removal of the thrombus is discussed above, the embodiments may instead be maneuvered within or proximate to the thrombus to puncture and/or break up the thrombus. Also, the thrombolytic agents applied to the capture segment may allow the capture segments to more readily enter/pass through the thrombus to puncture and/or break up the thrombus.

The foregoing has been a detailed description of illustrative embodiments of the invention. Various modifications and additions can be made without departing from the spirit and scope of this invention. For example, while specified materials are described, it is expressly contemplated that similar or superior materials may be employed if and when available for the described components of this invention. In particular, a variety of metals, polymers, composite, nano-materials and the like having desirable memory characteristics can be employed for capture segments and other components herein. Likewise, alternate techniques and materials can be employed for joining components. In addition further attachments can be provided to the devices described herein, with appropriate mounting hardware and locations to facilitate other, non-described procedures using the device. Accordingly, this description is meant to be taken only by way of example, and not to otherwise limit the scope of the invention.

What is claimed is:

1. A material-removal device, comprising:
an outer sheath including a proximal end and a distal end;
a core wire having a proximal end and a distal end, the core wire having an opposing actuator handle at the proximal end that extends from the proximal end of the sheath; and
a capture segment having a proximal end attached to the distal end of the sheath and a distal end attached at one side to the distal end of the core wire, the capture segment having a collapsed state and an expanded state creating a void at the distal end of the capture segment, the core wire being constructed and arranged so that applying axial movement to the handle causes the capture segment to controllably expand between the collapsed state and the expanded state for engaging a material within a blood vessel, wherein the capture segment remains in the expanded state for surrounding the material within the void and in the collapsed state for securing the material within the capture segment.

2. The device as in claim 1, wherein the material is a thrombus.

3. The device as in claim 1, wherein the capture segment is a braided mesh screen.

4. The device as in claim 1, wherein in the collapsed state, the capture segment is sized substantially similar to an outer diameter of the outer sheath.

5. The device as in claim 1, wherein in the expanded state, the capture segment is expanded to approximately an inner diameter of the blood vessel.

6. The device as in claim 1, wherein the capture segment in expanded state has a predetermined shape.

7. The device as in claim 1, wherein the capture segment comprises a material selected from the group consisting of: a braided material; a metallic material; and a non-metallic material.

8. The device as in claim 7, wherein the non-metallic material is selected from cloth and polymer fibers.

9. The device as in claim 1, wherein the capture segment comprises a metallic mesh selected from the group consisting of: nitinol, stainless steel, and cobalt-chromium alloy.

10. The device as in claim 1, wherein the capture segment comprises a radiopaque portion that is visible under fluoroscopy.

11. The device as in claim 1, further comprising:
a distal cap having an atraumatic leading edge at the distal end of the core wire.

12. The device as in claim 1, wherein the core wire comprises an extended portion beyond the distal end of the capture segment, the device further comprising:
an atraumatic spring having an atraumatic leading edge on the distal end of the extended portion of the core wire.

13. The device as in claim 1, wherein the distal end of the outer sheath comprises a flexible coil.

14. The device as in claim 1, wherein the capture segment is configured to be secured in one of either the expanded state or collapsed state.

15. The device as in claim 1, wherein the capture segment further comprises a material to attract a thrombus.

16. The device as in claim 15, wherein the material is selected from a group consisting of: an ionic charge, brushes, and filaments.

17. The device as in claim 1, further comprising:
a thrombus dissolving drug coated on the capture segment.

18. The device as in claim 1, wherein the core wire, outer sheath, and capture segment are configured to allow for infusion of drugs from the proximal end of the outer sheath to the distal end of the outer sheath.

19. A method for use with a material-removal device having an outer sheath including a proximal end and a distal end, the device further having a core wire having a proximal end and a distal end, the core wire having an opposing actuator handle at the proximal end that extends from the proximal end of the sheath, the device further having a capture segment having a proximal end attached to the distal end of the sheath and a distal end attached at one side to the distal end of the core wire, the capture segment having a collapsed state and an expanded state creating a void at the distal end of the capture segment, the method comprising:
applying axial movement to the handle to cause the capture segment to controllably expand between the collapsed state and the expanded state;
surrounding a material within a blood vessel within the void of capture segment in the expanded state; and
collapsing the capture segment to secure the material with the capture segment; and
moving the material by withdrawing the device.

20. The method as in claim 19, further comprising:
removing the material from the blood vessel with the capture segment in the collapsed state.

21. The method as in claim 19, further comprising:
securing the capture segment in one of either the collapsed state or the expanded state.

22. A method of making a material-removal device, comprising:
providing an outer sheath including a proximal end and a distal end;
inserting a core wire into the outer sheath, the core wire having a proximal end and a distal end, the core wire having an opposing actuator handle at the proximal end that extends from the proximal end of the sheath;
constructing a capture segment having a distal end and a proximal end, the capture segment having a collapsed state and an expanded state creating a void at the distal end of the capture segment;
attaching the proximal end of the capture segment to the distal end of the sheath; and
attaching the distal end of the capture segment at one side to the distal end of the core wire, the capture segment and the core wire being constructed and arranged so that applying axial movement to the handle causes the capture segment to controllably expand between the collapsed state and the expanded state for engaging a material within a blood vessel, wherein the capture segment is configured to remain in the expanded state to surround the material within the void and in the collapsed state to secure the material within the capture segment.
